(19) United States
(12) Patent Application Publication
(10) Pub. No.: US 2013/0041320 A1
(43) Pub. Date: Feb. 14, 2013

(54) DEVICE FOR GUIDING PERFUSION TUBE(S)

(71) Applicant: Julien ARN, Le Noirmont (CH)

(72) Inventor: Julien ARN, Le Noirmont (CH)

(73) Assignee: PERFUSAL SARL, Le Noirmont (CH)

(21) Appl. No.: 13/609,636

(22) Filed: Sep. 11, 2012

Related U.S. Application Data
(63) Continuation of application No. PCT/EP2011/054562, filed on Mar. 24, 2011.

(30) Foreign Application Priority Data
Mar. 25, 2010 (EP) 10157708.8

(51) Int. Cl. A61M 39/08; B29C 45/16

(52) U.S. Cl. 604/179; 264/255

(57) ABSTRACT

A device for guiding perfusion tube(s) (10), includes a wrist strap (11) with a closing member (14) so that it can be secured to a limb of a patient and bearing a guiding plate (12) for at least one perfusion tube (5, 6). The device is characterized in that the guiding plate (12) includes, in the body thereof, at least one first substantially U-shaped guiding groove (16) of a tube (5) and a second substantially straight guiding groove (17) extending in the longitudinal direction of the wrist strap (11) and arranged tangentially to the first guiding groove (16) so that a tube (5) can be partially arranged in one of the first guiding grooves and partially in the other guiding groove.

The present application is a continuation of international application PCT/EP2011/117363, filed on Mar. 24, 2011, the content of which is hereby enclosed by reference. It claims priority of European Patent Application EP10157798 filed on Mar. 25, 2010, the content of which is hereby enclosed by reference.

### TECHNICAL FIELD

The present invention relates to a device for guiding perfusion tube(s), comprising a wrist strap with a closing member so that it can be secured to a limb of a patient and bearing a guiding plate for at least one perfusion tube.

Such a device is designed to improve the comfort of a patient having notably to endure a prolonged perfusion as well as its safety, due to the fact that it makes it possible to limit the risk of the perfusion being accidentally torn off. This device also aims to be beneficial for the medical staff using it.

The present invention also relates to a method for manufacturing such a device.

### STATE OF THE ART

Many devices of this type have been disclosed in the state of the art, such as for example in U.S. Pat. No. 5,916,199 of 29 Jun. 1999. This document describes a device for guiding a perfusion tube, comprising a wrist strap of the hook and loop fastening type (Velcro, registered trademark), on which a guiding plate is slipped. The latter comprises a base and a cover connected to the base so as to be able to pivot between an opened and a closed position. The base and the cover each comprise a U-shaped channel, with the channels being configured so as to cooperate one with the other in the closed position of the cover so as to define a guiding recess for a perfusion tube.

It must however be noted that this device has a considerable number of disadvantages. Its structure is complex, which entails not only a costly manufacturing process, but also a relative slowness in it being fastened into place on a patient's limb. Indeed, the guiding plate must be threaded onto the wrist strap before the latter is integrally united with the limb of a patient on a perfusion, following which the tube must be placed in the channel of the base before proceeding with closing the plate by pivoting the cover.

Furthermore, it will be noted that this device is not of interest when the perfusion needle is inserted for example in the hand in the direction going from the shoulder towards the hand (i.e. in the configuration inverted to that illustrated in FIG. 1 of this patent). In this case, only an additional guiding device, also described in this document and having a rectilinear guiding recess, can be implemented to ensure the tube is guided towards the upper part of the patient's body.

Patent application WO2009/137607 A1, published on 12 Nov. 2009, describes another type of guiding device for perfusion tube, whose fastening into place of the tube on the guiding members is simplified relatively to the device described here above. The structure disclosed for the tube guiding members further provides greater flexibility for adapting the guiding of the tube to the implantation mode of the perfusion on the patient's limb.

However, the number of constituting elements of the device as well as their shapes makes the manufacturing and assembly process of the device complex and costly. It must be noted that use of these devices is a question of mass production. Each hospital must be able to get equipped with such devices in a quantity greater than the number of beds it contains. Consequently, the purchase price of a device of this type for a hospital should be as low as possible, and if possible adapted to single use.

Furthermore, it will also be noted that one and the other of the prior art devices described here above have an additional disadvantage, also related to the corresponding structures. These devices in fact comprise many crevices or recesses in which soiling can easily become lodged, which makes them unhygienic.

U.S. Pat. No. 5,147,320 describes a catheter affixing and anchoring device comprised of an elastomer plate comprising a tunnel and a channel for fastening a perfusion tube. A junction enables the tube to be placed in two different ways on the plate. However, this device enables only a single perfusion tube to be fastened, whereas it is often useful to affix two of them. Furthermore, in all fastening embodiments, the perfusion tubes are parallel to the wrist and thus arrive directly against the back of the hand, which is uncomfortable and hampers the wrist's movements. The elastomer plate moreover becomes deformed quite easily, so that the tubes can involuntarily leave the groove when the plate is folded. In order to reduce this risk, one portion of the insertion groove is configured as a tunnel, which however makes it more difficult to insert the tube.

WO2008072946 describes another device for securing a catheter directly onto the back of the hand. This position is uncomfortable and, again, the tubes leaving the device parallel to the fingers hamper the wearer's movements. This device furthermore does not enable several perfusion tubes to be affixed.

Similarly, WO9956802 describes a device designed for a single perfusion tube, whose two ends are parallel to the patient's wrist.

U.S. Pat. No. 4,453,933 describes a device enabling several tubes to be guided, but again all parallel to the arm.

### AIMS OF THE INVENTION

A main aim of the present invention is to remedy the disadvantages of the devices for guiding perfusion tubes known in the prior art, by proposing such a device that can be used in an easy and flexible manner, enabling it to be used on a large scale in a hospital environment.

To this effect, the present invention concerns more particularly a device for guiding perfusion tube(s) of the type mentioned above, characterized in that the guiding plate comprises, in the body thereof, at least a first substantially U-shaped guiding groove of a tube and a second substantially straight guiding groove extending in the longitudinal direction of the wrist strap and arranged tangentially to the first guiding groove, so that a tube can be partially arranged in one of the first guiding grooves and partially in the other guiding groove, in such a way that a tube can optionally be placed in said grooves so as to exit from said plate in a direction parallel or perpendicular to the patient's wrist when the guiding plate is worn by the patient.

Thanks to its characteristics, the guiding device according to the invention has a simple structure involving an easy and flexible fastening into place of the tube, i.e. that can be easily adapted to the mode for implanting the perfusion on the patient.

This device can further comprise a place for inserting or affixing there a patient identification label and/or an electronic chip enabling information on the patient to be entered there for the attention of the medical staff.

The invention is based notably on the observation that different arrangements of the perfusion tubes on the patient's wrist can be conceived and that each of these arrangements provides a certain number of advantages. For example, some types of perfusion require a single tube whilst others require two distinct tubes. Some tubes are provided with a valve close to the plate and which can impede the patient, other interventions do not require such a valve or allow it to be placed elsewhere. Some patients or nurses prefer the tubes to be placed so that they exit from the plate in a direction parallel to the wrist, whilst in other circumstances it is more comfortable or convenient for at least one of the ends of the tube to exit from the plate in a direction perpendicular to this wrist.

Preferably, the guiding plate comprises at least a third guiding groove substantially parallel to the second guiding groove. This particular characteristic enables the guiding of an additional tube to be achieved, notably in the case of a perfusion with two simultaneous entries.

The second groove and the third groove are advantageously oriented so that said tubes exit from said plate in a direction perpendicular to the patient's wrist.

The first substantially U-shaped guiding groove makes it possible to place a tube whose two ends exit from the plate in a direction substantially parallel to the patient's wrist and in the direction of the patient's forearm (i.e. towards the side opposite to the patient's hand). As the base of the U of the first groove is arranged tangentially to the second groove, a tube can also be partially arranged in the first U-shaped guiding grooves and partially in the second groove, so that one of the two ends of this tube (the one that is in the first groove) exit from the plate in a direction substantially parallel to the patient's wrist whilst the other end engaged in the second groove exits from the plate in a direction substantially perpendicular to the patient's wrist.

According to a preferred embodiment, each of the guiding grooves has, in cross-section from the free surface of the guiding plate, two substantially parallel walls joining in a curve substantially in the shape of a semi-circle. Furthermore, each of the grooves comprises at least one region having at least some thickening provided on at least one of the walls, close to the free surface of the guiding plate, and defining a constriction.

Thus, fastening a tube into place in a groove is very simple since it is necessary only to insert it by pressure, with the tube thus being subjected to a slight temporary compression when passing through the constriction. It is thus possible to avoid the disadvantages of closed tunnel grooves, into which it is difficult to insert the tubes.

In a particularly advantageous manner, the wrist strap and the guiding plate, and even the closing member, are made of a single piece.

Thanks to these characteristics, not only is the manufacture process of the inventive device simplified as compared with the manufacture processes of the prior art devices, but it is furthermore possible to control accurately the shape of the device, notably to avoid the formation of crevices into which dirt could become lodged during subsequent use.

In a similar way, the closing member should advantageously have the simplest structure possible, both for reasons of manufacture as for the cleanliness of the device. It is thus possible to provide a button designed to cooperate with a hole from a plurality of holes provided in the wrist strap.

Furthermore, the particular characteristics of the device that have just been enumerated here above can be achieved by implementing a manufacturing process comprising the steps of:

a) providing an injection mold corresponding to the desired shape for the wrist strap, the guiding plate and the closing member;

b) providing an overmolding system enabling materials of different rigidity to be overmolded and/or assembled in order to mold said wrist strap, said closing member, and to overmold onto said wrist strap the guiding plate comprising at least a first substantially U-shaped groove for guiding a tube and a second substantially straight guiding groove extending in the longitudinal direction of the wrist strap and arranged tangentially to the first guiding groove, so that a tube can optionally be placed in said grooves so as to exit from said plate in a direction parallel or perpendicular to the patient's wrist;

c) performing an injection molding by means of the mold and of the bonding system to form the guiding device.

### BRIEF DESCRIPTION OF THE FIGURES

Other characteristics and advantages of the present invention will appear more clearly when reading the following detailed description of a preferred embodiment, with reference to the attached drawings given by way of non limiting examples and wherein:

FIG. 1 represents a simplified front view of an example of execution of the guiding device according to a preferred embodiment of the present invention, in a first configuration;

FIG. 2 represents a similar view to that of FIG. 1, in a second configuration;

FIG. 3 represents a simplified front view of a guiding device according to a preferred embodiment of the present invention;

FIG. 4 represents a simplified side view of the guiding device of FIG. 3; and

FIG. 5 represents a simplified rear view of the guiding device of FIG. 3;

FIG. 6 illustrates a simplified perspective view of a guiding device.

### EMBODIMENT(S) OF THE INVENTION

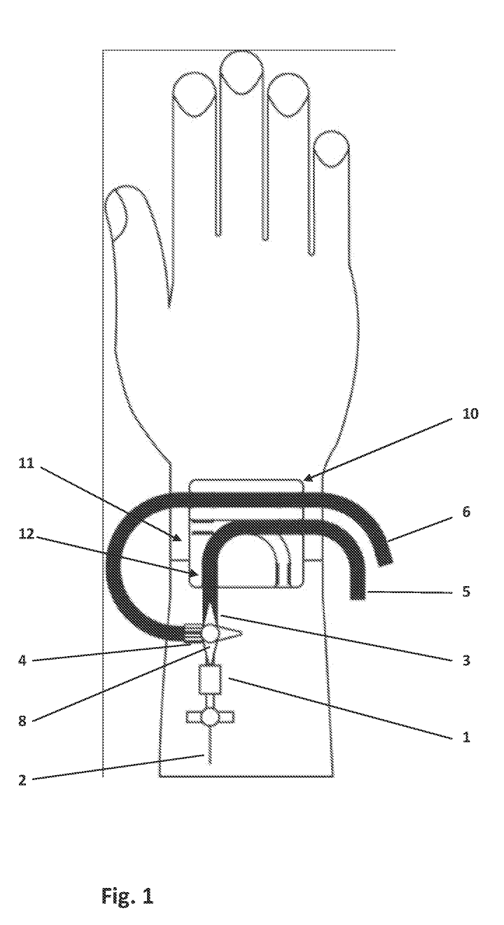

FIG. 1 represents a simplified front view of an example of execution of the guiding device according to a preferred embodiment of the present invention, in a first configuration.

More precisely, FIG. 1 illustrates the fastening into place of a perfusion 1 on the forearm of a patient. The needle 2 is implanted in the forearm in the direction from the hand towards the shoulder. In this way, the inlet of the tube or tubes feeding the liquid for infusion is situated on the side of the hand.

In the example represented here, the perfusion comprises first and second inlets 3 and 4, each being connected to a tube 5, 6.

The second inlet 4 is provided with a valve 8 and is oriented towards the patient's body, the first inlet being oriented toward the patient's hand.

The patient wears a device for guiding tubes 10 according to a preferred embodiment of the present invention. The latter comprises a wrist strap 11 bearing a tube guiding plate 12. It arises from FIG. 1 that the tubes 5 and 6 are fixed into place on the guiding plate 12 and are guided to be oriented towards the outside of his arm.

Thanks to this guiding and because the tubes are held securely on the guiding plate, the patient enjoys a greater freedom of movement than with certain tube guiding devices of the prior art. In particular, it will be noted that the guiding device according to the present preferred embodiment advantageously enables two tubes to be guided simultaneously, which was note the case of the prior art devices presented further above.

This device further advantageously has a place 11b for inserting or affixing there a patient identification label. An electronic chip can also be inserted into the device, for example inside the guiding plate, for storing medical information relative to the patient that can be read by a RFID reader.

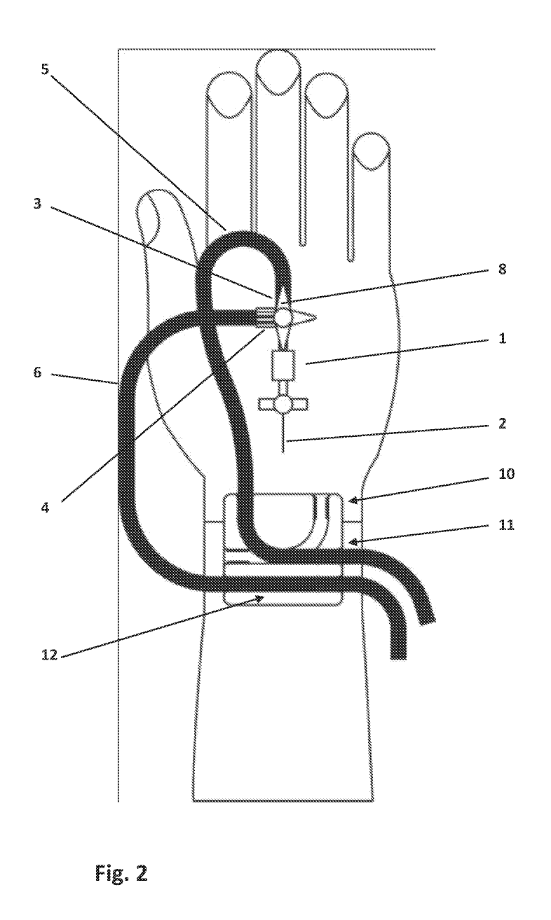

FIG. 2 represents a view similar to that of FIG. 1, in a second configuration.

More accurately, FIG. 2 illustrates the fastening into place of the perfusion 1 directly onto the patient's hand. The needle 2 is implanted in the hand in the direction from the hand towards the shoulder. In this way, the inlet of the tube or tubes feeding the liquid for infusion is situated on the side of the hand, as in the case of FIG. 1.

However, in contrast to the configuration of FIG. 1, the tubes 5 and 6 do not extend in the direction of the patient's wrist from the perfusion 1 but in the direction of his fingers.

Thus, the tubes 5, 6 can advantageously be placed so as to return in the direction of the wrist where the guiding device according to the present invention is laid out, from the side of the hand situated close to the patient's body. The tubes can then be fixed into place on the guiding plate 12 so that they are then oriented towards the outside of the patient's arm, in the same way as in the case of the configuration of FIG. 1.

It will be observed that the tube guiding device 10 is placed in opposite direction in the configuration of FIG. 2 with reference to the configuration of FIG. 1, its reversal between one and the other configuration making it possible to achieve the same advantageous final result, as previously explained in relation to FIG. 1.

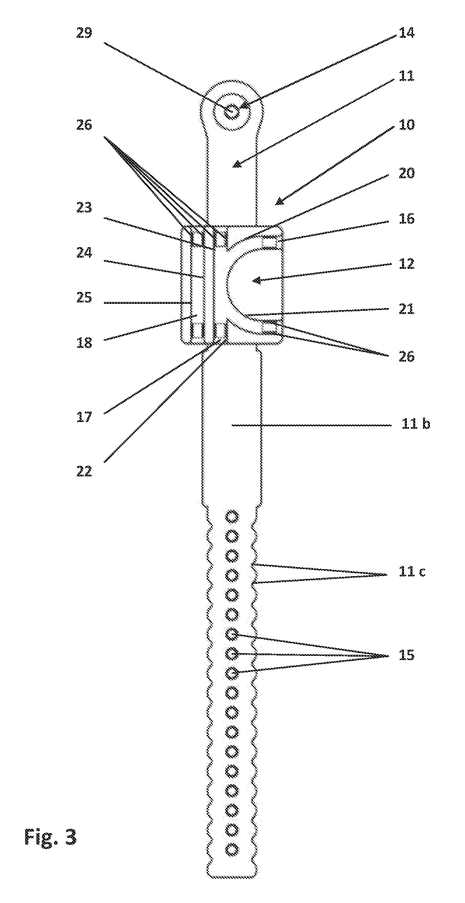

A guiding device 10 according to a preferred embodiment of the present invention will be described in more detail on the basis of FIGS. 3, 4 and 5.

FIG. 3 represents a simplified front view of this guiding device 10.

The wrist strap 11 comprises a closing member 14 made here in the shape of a button, situated at a first extremity of the wrist strap and designed to cooperate with holes 15 provided close to the second extremity of the wrist strap, in a conventional manner. It will be noted that such a closing member exhibits great simplicity both in its manufacturing as well as in its use. Furthermore, the wrist strap 11 has semi-circular undulations 11c on its lateral edges, enabling it to be adjusted to the diameter of the wrist by simply being cut and avoiding that a sharp angle should injure or hamper the patient.

The guiding plate 12 is arranged in the medial region of the wrist strap 11.

It arises clearly from FIG. 3 that the guiding plate comprises a first substantially U-shaped tube-guiding groove 16 as well as a second substantially straight guiding groove 17, extending in the longitudinal direction of the wrist strap and arranged tangentially to the first guiding groove so that the two grooves communicate on with the other. Thanks to this characteristic, it is possible for a tube to be partially arranged in a first of the guiding grooves and, simultaneously, partially in the other guiding groove. Such an arrangement arises from the configurations represented in FIGS. 1 and 2, for the first tube 5.

Furthermore, according to the preferred embodiment as illustrated in the figures, the guiding plate 12 comprises a third guiding groove 18, substantially parallel to the second guiding groove 17, whose purpose is to ensure that an additional tube is guided, as the tube 6 illustrated in FIGS. 1 and 2. The guiding plate is advantageously rigid, which makes it possible to prevent the tubes from being chased outside of the grooves when the plate becomes deformed. The guiding plate is preferably more rigid than the wrist strap 11, which can be made of another material or of the same material processed differently. A limited flexibility can however be provided in order to improve the guiding plate. Preferably, the plate is more rigid than the perfusion tubes that can thus easily follow the plate's deformations.

The advantages when using the guiding device according to this preferred embodiment appear clearly from FIGS. 1 to 3, notably the fact that it can be used in one direction as well as in the other, depending on the area in which the perfusion is implanted in the patient's limb.

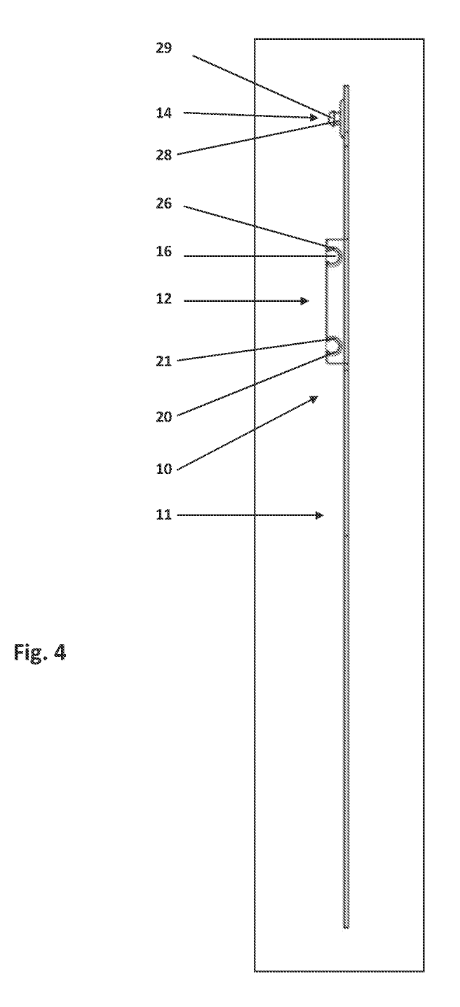

FIG. 4 represents a simplified side view of the guiding device of FIG. 3, from which, in combination with FIG. 3, it can be seen that each of the grooves 16, 17 and 18 has two walls 20 and 21, 22 and 23, 24 and 25, substantially parallel and joining two-by-two in a curve having a substantially semi-circular shape.

Of course, it is alternatively possible to provide that the walls are slightly curved inwards, as arises from FIG. 4 for the walls 20 and 21, without stepping outside the frame of the present invention. According to this alternative, the first guiding groove 16 has a cross-section with a partly circular outline.

In one case as in the other, it is preferable to further provide that each of the grooves comprises at least one region having at least some thickening 26 provided on at least one of the walls, close to the free surface of the guiding plate 12, and defining a constriction. In particular, according to the present preferred embodiment, each guiding groove is provided with four thickenings 26 to ensure the tube guided along each groove is securely held.

As previously mentioned, the fastening into place of a tube in a groove is very easy since it is necessary only to insert it by pressure, with the tube thus being subjected to a slight temporary compression when passing through the constriction.

It will be further noted that the inventive device has the additional advantage of ensuring that the tubes are well visible at all times, which makes it possible to check that they are not obstructed when a product is being administered to a patient.

Furthermore, it arises from FIG. 4 that the closing button 14 comprises here a substantially cylindrical base 28 surmounted by a semi-sphere 29 designed to ensure that the button is held in one of the holes 15.

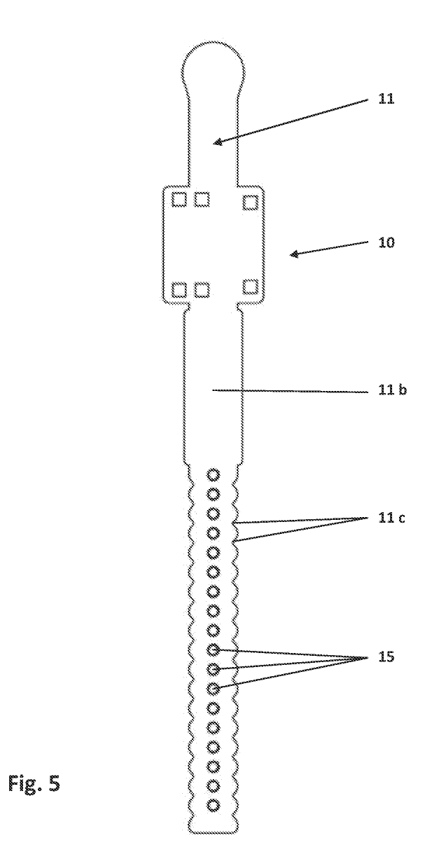

FIG. 5, which represents a simplified rear view of the guiding device of FIG. 3, further enables the structural simplicity of the guiding device according to the present preferred embodiment of the invention to be appreciated.

It appears indeed from FIG. 5 that the back of the device is perfectly smooth and has no opening, apart from the closing holes 15 and the manufacturing holes 30, that could cause dirt to accumulate. The device is further preferably designed for single-use to ensure maximum hygiene. Devices that can be sterilized can also be conceived.

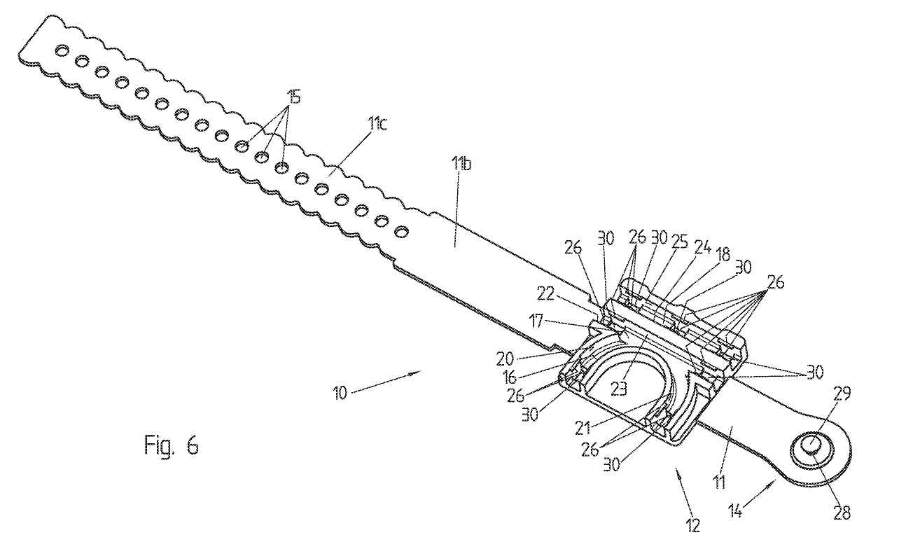

FIG. 6 illustrates a perspective view of a guiding device according to one embodiment of the invention. The elements that are identical or similar to those already described in relation to the above embodiments bear the same reference numbers. This figure shows in particular the thickenings 26 that enable the tubes to be held after their insertion into the grooves; the tubes are slightly deformed in order to pass the constriction between two opposing thickenings and regain their shape when they are lodged at the bottom of the groove. The holes 30 are openings designed for the molding and unmolding of these thickenings 26. The border of the grooves is constituted by relatively fine protruding ribs, so that the portions of the wrist strap outside the grooves are thinner than these edges; this characteristic makes is possible to economize on material and to better control the material shrinkage during hardening.

The wrist strap 11 can advantageously be made of a single piece of plastic material, preferably by molding which enables a manufacturing cost compatible with a single-use device to be achieved. It is of course possible to use several plastic materials having different mechanical properties adapted to the constraints that the different parts of the device are necessarily subjected to. It is for example possible and desirable to use for the plate a more rigid plastic than for the wrist strap. The wrist strap is advantageously made of a non-tinted antiallergenic material.

The color of the wrist strap can also be indicated as a marker to indicate the patient's pathology or the hospital service in charge. The invention thus also relates to assortments of guiding devices of different colors.

More precisely, it can be provided for that the guiding device according to the invention is manufactured by implementing a manufacturing process comprising the steps of:

a) providing with an molding or injection molding system enabling several materials or identical materials of different rigidity to be overmolded and/or assembled, with the molding system corresponding to the desired shape for the wrist strap, the guiding plate and the closing member;

b) molding in said molding system the wrist strap and the closing member and overmolding onto said wrist strap said guiding plate of a more rigid material than that used for the wrist strap.

In another embodiment, the mold comprises at least one drawer for providing, in the body of the guiding plate, at least one first substantially U-shaped tube-guiding groove and a second substantially straight guiding groove, extending in the longitudinal direction of the wrist strap and arranged tangentially to the first guiding groove.

It is also possible to provide that the mold is arranged so as to enable the simultaneous molding of the closing member 14.

The mold also enables thickenings 26 to be molded thanks to portions of the mold that traverse openings 30 through the plate, opposite these thickenings.

The one skilled in the art can provide for slits to be made in the thickness of the wrist strap to enable the patient's skin to breathe, without stepping outside the frame of the present invention. In a similar manner, it will also be possible to provide that instead of being cylindrical, the holes 15 are widened on the side through which the button 14 is to be inserted, in order to make this insertion easier without drawbacks as regards the holding quality of the button once it is lodged in a given hole.

The one skilled in the art will obviously find it advantageous to execute the device according to the present invention in several sizes adapted to different morphologies of patients, such as for example at least one child size and one adult size. Furthermore, it will also be possible to provide for one or two loops to be arranged on the wrist strap to hold its free extremity when it is closed. These loops can be mobile or fixed and can possibly also be made directly during molding of the wrist strap.

1. A device for guiding perfusion tube(s), comprising a wrist strap with a closing member arranged for securing it to a limb of a patient and bearing a guiding plate for at least one perfusion tube,
wherein said guiding plate comprises, in the body thereof, at least one first substantially U-shaped guiding groove of a tube and a second substantially straight guiding groove extending in the longitudinal direction of said wrist strap and arranged tangentially to said first guiding groove so that a tube can be partially arranged in a first of said guiding grooves and partially in the second guiding groove, in such a way that a tube can optionally be placed in said grooves so as to exit from said plate a in a direction parallel or perpendicular to the patient's wrist,
said guiding plate further comprising a third guiding groove substantially parallel to said second guiding groove, so that two tubes can be placed simultaneously onto said guiding plate,
wherein said second groove and said third groove are oriented so that said tubes exit from said plate in a direction perpendicular to the patient's wrist,
and wherein said first substantially U-shaped guiding groove makes it possible to place a tube whose two ends exit from the plate in a direction substantially parallel to the patient's wrist.

2. Device according to claim 1, wherein said first substantially U-shaped guiding groove makes it possible to place a tube at least one end of which exits from the plate in the direction of the patient's forearm.

3. Device according to claim 1, wherein said first substantially U-shaped guiding groove makes it possible, in collaboration with said second guiding groove to place a tube, one end of which exits from the plate in a direction substantially parallel to the patient's wrist and the other end of which exits from the plate in a direction substantially perpendicular to the patient's wrist.

4. Device according to claim 1, wherein each of said guiding grooves has, in cross-section from the free surface of the guiding plate, two substantially parallel walls joining in a curve substantially in the shape of a semi-circle and wherein each of said grooves comprises at least one region having at least some thickening provided on at least one of said walls, close to the free surface of said guiding plate, and defining a constriction.

5. Device according to claim 4, comprising at least one hole through the plate opposite one said thickness and enabling the molding and unmolding of said thickness.

6. Device according to claim 1, wherein said wrist strap and said guiding plate are made of one piece.

7. Device according to claim 1, wherein said closing member comprises a button designed to cooperate with a hole from a plurality of holes provided in said wrist strap, said button being formed of one piece with said wrist strap and said plate.

8. Device according to claim 1, wherein said wrist strap has a plurality of slits in the thickness thereof to enable the patient's skin to breathe.

9. Device according to claim 1, with a smooth place for sticking a patient identification label.

10. Device according to claim 1, with an electronic chip enabling patient data to be stored.

11. Device according to claim 1, wherein the lateral edges of the wrist strip have undulations to facilitate the cutting of the wrist strap to the desired length.

12. Device according to claim 1, said guiding plate being overmolded onto said wrist strap.

13. Manufacturing process of a device for guiding perfusion tube(s), comprising a wrist strap with a closing member so that it can be secured to a limb of a patient and bearing a guiding plate for at least one perfusion tube, comprising the steps consisting of:
a) providing an injection mold corresponding to the desired shape for said wrist strap, for a closing member of said wrist strap and for said guiding plate;
b) injecting into said system said wrist strap and/or said closing member in a first material;
c) overmolding into said system said guiding plate in a second material, so that a tube can optionally be placed in said grooves so as to extend from said plate along a direction parallel or perpendicular to the patient's wrist.

14. Method according to claim 13, wherein said mold is arranged for also enabling said closing member to be molded simultaneously.

15. Method according to claim 14, wherein said mold is arranged for enabling thickenings to be molded in said grooves, thanks to holes traversing said plate.
